(19) United States
(12) Plant Patent Application Publication
(10) Pub. No.: US 2020/0205331 P1
(43) Pub. Date: Jun. 25, 2020

(54) GRAPE PLANT NAMED 'PINOT ISKRA'

(50) ;

(71) Applicants: UNIVERSITÀ DEGLI STUDI DI UDINE, Udine (IT); ISTITUTO DI GENOMICA APPLICATA, Udine (IT)

(72) Inventors: Simone Diego Castellarin, Vancouver (CA); Guido Cipriani, Faedis (IT); Gabriele Di Gaspero, Cividale del Friuli (IT); Michele Morgante, Tricesimo (IT); Enrico Peterlunger, Codroipo (IT); Raffaele Testolin, Udine (IT)

(73) Assignees: UNIVERSITÀ DEGLI STUDI DI UDINE, Udine (IT); ISTITUTO DI GENOMICA APPLICATA, Udine (IT)

(21) Appl. No.: 16/602,637

(22) Filed: Nov. 12, 2019

(30) Foreign Application Priority Data
Dec. 20, 2018 (EU) 20183516

(51) Int. Cl. A01H 6/88

(52) U.S. Cl. PLT/207

(57) ABSTRACT

A new grape variety distinguished by its resistance to downy mildew and powdery mildew, strong blistering on the upper side of the leaf blade, early-medium harvesting time (end of August in northeastern Italy), and semi-erect shoot attitude.

### CROSS REFERENCE TO RELATED APPLICATIONS

This application claims the benefit of priority from EU Community Plant Variety Rights (CPVR) Application No. 20183516, filed Dec. 20, 2018.

Latin name of the genus and species of the plant claimed: Vitis vinifera L.

Variety denomination: ‘PINOT ISKRA’.

### BACKGROUND

The present application relates to a new and distinct variety of grape named ‘PINOT ISKRA’. Our new plant resulted from a controlled cross and is a selection from crossing SK-00-1/7 (unpatented) as the seed parent with Pinot Blanc (unpatented) as the pollen parent in 2005. The resulting plant was selected in 2013 when growing in a cultivated area in Udine, Italy.

### SUMMARY

The ‘PINOT ISKRA’ variety is distinguished from other grape varieties due to the following unique combination of characteristics: resistance to downy mildew and powdery mildew, strong blistering on the upper side of the leaf blade, early-medium harvesting time (end of August in northeastern Italy), and semi-erect shoot attitude. A comparison of the new variety to its male parent (Pinot Blanc, the most similar variety) is provided below.

| Charac- | | Male Parent |
|---|---|---|
| teristic | ‘PINOT ISKRA’ | Pinot blanc |
| vigour | Medium-high | Medium |
| growth | Semi-erect | Horizontal |
| habit | | |
| leaf | Very small to small, dark | Medium, dark green color |
| | green color (upper surface) | (upper surface) N134A, |
| | N134A, very few hairs in | very few hairs in both |
| | both surfaces, pentagonal | surfaces, pentagonal shape, |
| | shape, medium teeth, | small teeth, medium blister- |
| | strong blistering of upper | ing of upper side of blade |
| | side of blade | |
| cluster | low weight, cylindrical | low weight, cylindrical with |
| | with one or two middle | one or two wings, compact, |
| | size wings, compact, | broad ellipsoid berry, berry |
| | globose berry, berry skin | skin with yellow-green color, |
| | with yellow-green color, | soft flesh, neutral taste, no |
| | soft flesh, neutral taste, | flesh coloration |
| | no flesh coloration | |
| harvesting | Early-medium [end of | Medium [early September |
| time | August (Middle Friuli, | (Middle Friuli, northeastern |
| | northeastern Italy)] | Italy)] |
| resistances | Resistant to downy | No resistance to downy |
| | mildew and to powdery | mildew or to powdery |
| | mildew | mildew |

Asexual reproduction of this new variety by vegetative cuttings shows that the foregoing characteristics come true to form, are firmly fixed, and are established and transmitted through succeeding propagations.

Certain characteristics of this variety may change with changing environmental conditions (such as photoperiod, temperature, moisture, soil conditions, nutrient availability, or other factors). Color descriptions and other terminology are used in accordance with their ordinary dictionary descriptions, unless the context clearly indicates otherwise. Color designations (hue/value/chroma) are made with reference to The Royal Horticultural Society (R.H.S.) Colour Chart, 5^{th} edition, London, England, 2007.

### BRIEF DESCRIPTION OF THE DRAWINGS

FIG. 1 is a photograph showing a shoot a leaf of the new variety.

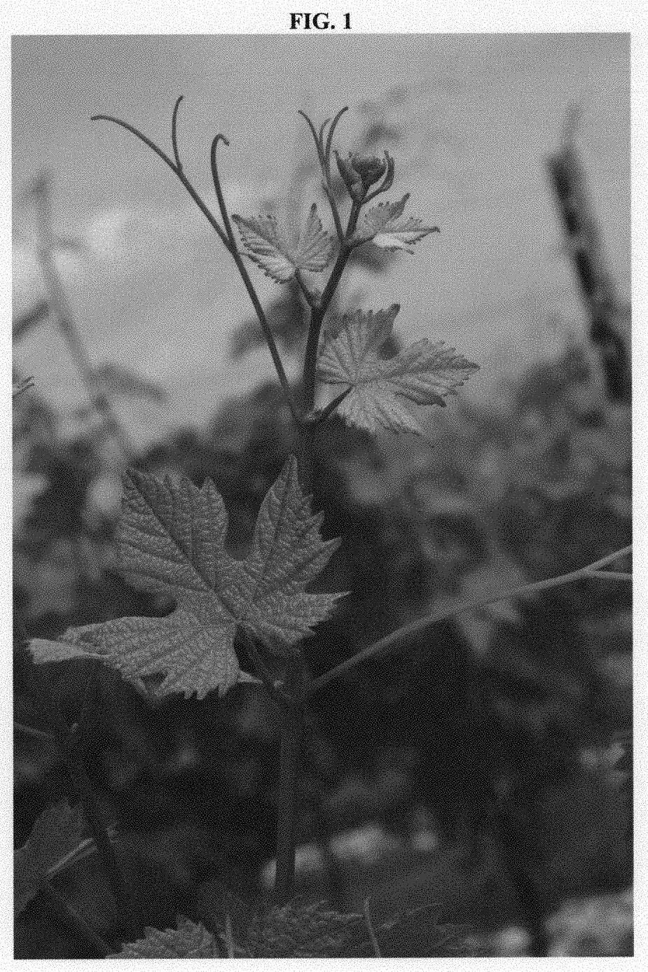

FIG. 2 is a photograph showing a leaf of the new variety.

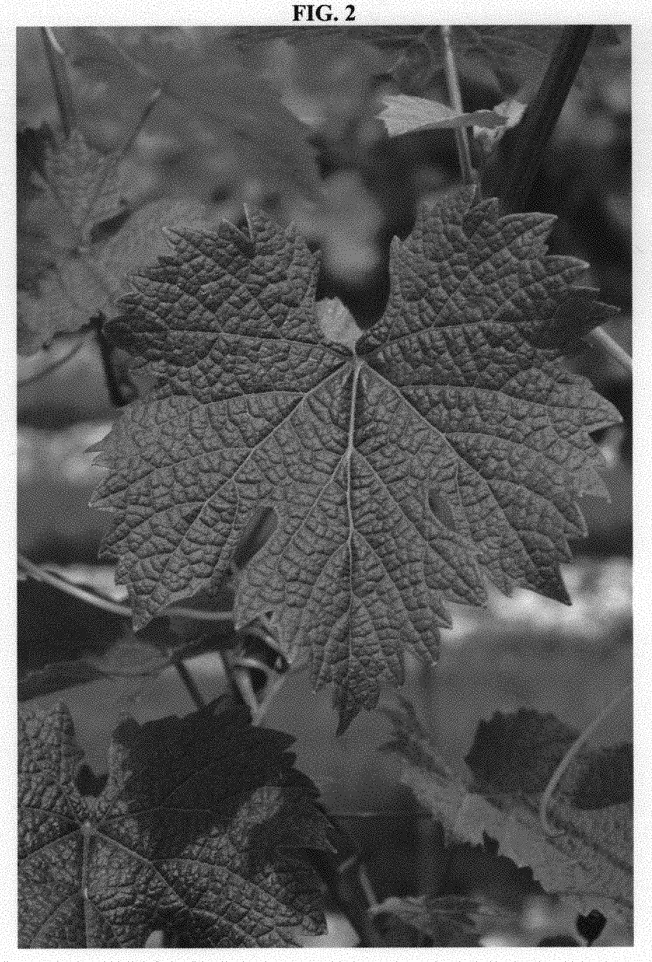

FIG. 3 is a photograph showing fruit of the new variety.

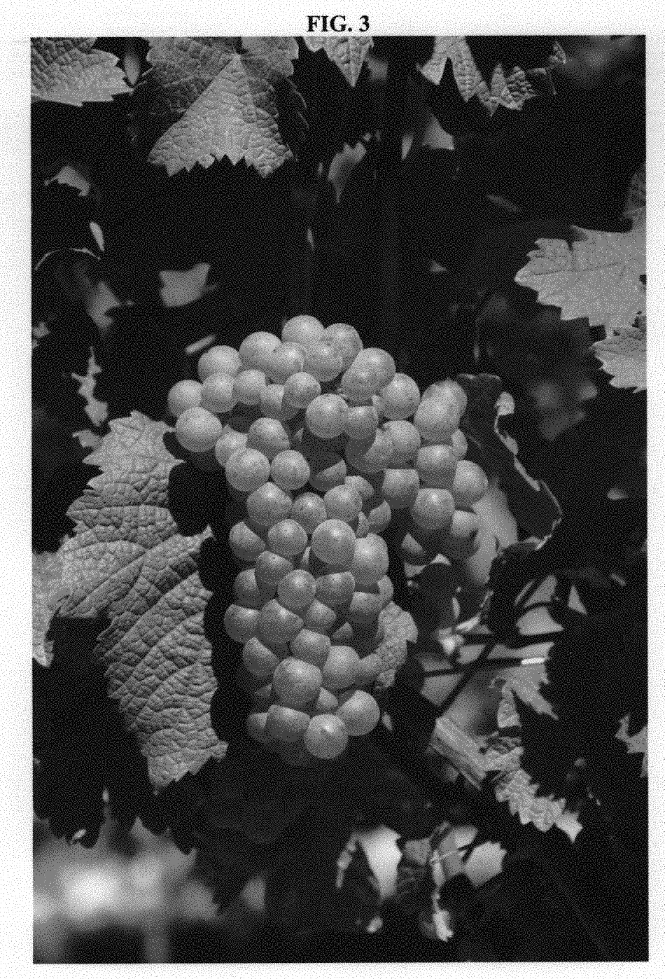

FIG. 4 is a photograph showing a plant of the new variety.

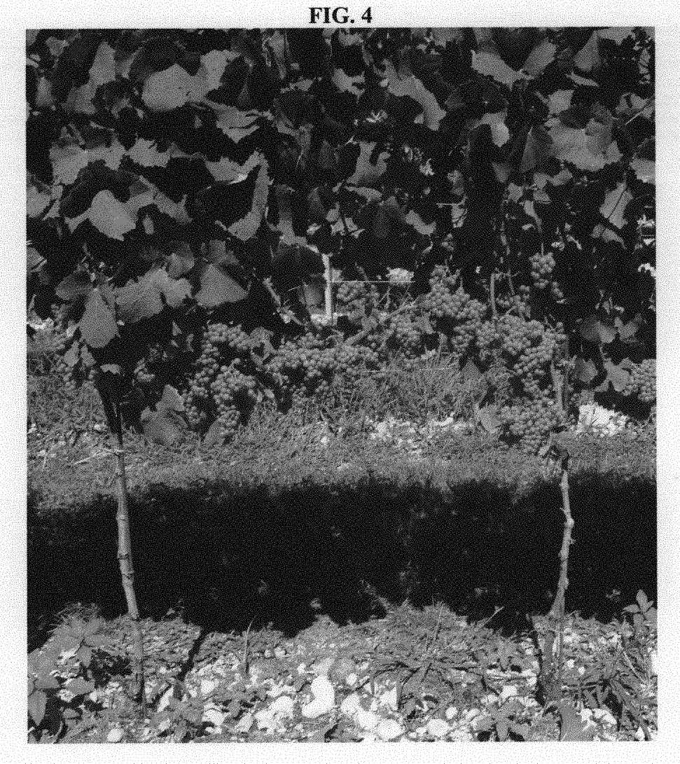

The color photograph shows typical specimens of the new variety and depict the color as nearly true as is reasonably possible to make the same in a color illustration of this character. It should be noted that colors may vary, for example due to lighting conditions at the time the photograph is taken. Therefore, color characteristics of this new variety should be determined with reference to the observations described herein, rather than from the photograph alone.

### DETAILED DESCRIPTION

### Botanical

The following detailed description of the ‘PINOT ISKRA’ variety is based on observations of asexually reproduced progeny. The observed progeny are plants which were 3-5 years of age. The following detailed description concerns the plants growing in an open field taken in Rauscedo, Italy in 2016-2018. The original plant and progeny have been observed growing in a cultivated area in Rauscedo, Italy, with medium texture soil that is rich in skeleton and alluvial in nature. Temperatures in Rauscedo, Italy range from a high of 29° C. to a low of −2° C. Average rainfall is 822 mm per year, with an average rainfall during the growing season (April-September) of 453 mm.

The chart used in the identification of colors described herein is The R.H.S. Colour Chart, 5^{th} edition, except where general color terms of ordinary significance are used. The color values were determined in August-September 2018 under natural light conditions in Rauscedo, Italy.
Scientific name: Vitis vinifera L.
Parentage:
   Seed parent.—Sk-00-1/7.
   Pollen parent.—Pinot Blanc.
Plant:
   Vigor.—Medium-high.
   Growth habit.—Horizontal.
Shoot:
   Opening of the shoot tip.—Fully open for young shoot.
   Distribution of the anthocyanin coloration of the prostrate hairs of the shoot tip.—Piping.
   Density of prostate hair on the shoot tip.—Low.
   Attitude (before tying).—Semi-erect.
   Color of the dorsal side of internodes.—Green 140B and red 53A.
   Color of the ventral side of internodes.—Green 140B and red 53A.
   Distribution of anthocyanin coloration on the bud scales.—Absent.
   Number of consecutive tendrils.—2 or less.
Leaves:
   Shape.—Pentagonal.
   Teeth.—Medium.
   Blistering.—Strong blistering of upper side of blade.
   Size.—Very small to small.
   Young leaf.—Color of upper surface is green 134A, with medium prostrate hairs between main veins on the lower side of blade.
   Mature leaf.—Color of upper surface is dark green N134A, with sparse prostrate hairs between main veins on the lower side of blade.
   Mature leaf.—Five lobes.
   Area of anthocyanin coloration of main veins on upper side and lower of mature blade.—Up to the 1^{st} bifurcation.
   Goffering of mature blade.—Medium.
   Shape of base of petiole sinus.—Brace-shaped.
   Petiole sinus base limited by vein.—Not limited.
   Density of prostrate hairs on petiole.—Low.
   Density of erect hairs on petiole.—None or very low.
   Depth of upper lateral sinuses.—Medium.
Flowers: Fully developed stamens and fully developed gynoecium
   Insertion of 1^{st} inflorescence.—3^{rd} and 4^{th} node.
   Number of inflorescences per shoot.—1 to 3.
Fruit:
   Cluster.—Low weight, cylindrical with one or two middle size wings, compact.
   Time of beginning of berry ripening.—Early.
   Berry shape.—Globose.
   Berry length.—About 13 mm.
   Berry width.—About 13 mm.
   Thickness of berry skin.—Medium-to-thick.
   Berry hilium.—Visible.
   Color of skin (without bloom).—Yellow green (154C).
   Texture.—Soft flesh.
   Anthocyanin coloration of flesh.—Absent or very weak.
   Flavor.—None/neutral.
   Formation of seeds.—Complete.
   Harvest time.—Early-medium (end of August in northeastern Italy).
   Bunch length (peduncle excluded).—Short-to-medium.
   Bunch width.—Narrow.
   Bunch density.—Dense.
   Length of peduncle of primary bunch.—Short-to-medium.
   Lignification of peduncle.—Up to about the middle to more than the middle.
Must characteristics:
   Sugars (Brix).—20.6.
   pH.—3.3.
   Total acidity.—6.1 g/l.
   Tartaric acid.—5.52 g/l.
   Malic acid.—1.76 g/l.
Production characteristics:
   Clusters per shoot.—1.7.
   Grape production.—3133 g/plant.
   No. of bunches/vine (at harvest).—27.6.
   Average weight of the bunch.—168 g.
   Average berry weight.—1.53 g.
   Pruning wood weight.—610 g/plant.
   Index of ravaz.—5.13.
Wine produced from grapes:
   Total acidity.—5.7 g/l.
   Tartaric acid.—3.3 g/l.
   pH.—3.2.
   Net extract.—19.1 g/l.
   Alcohol.—13.9 g/l.
   Volatile acidity.—0.3 g/l.
   Reducing sugars.—1.4 g/l.
Molecular analysis:

| VVS2 | | VVMD5 | | VVMD7 | |
|---|---|---|---|---|---|
| N + 10 | N + 28 | N + 6 | N + 10 | N + 8 | N + 26 |
| VVMD25 | | VVMD27 | | VVMD28 | |
| N + 6 | N + 14 | N + 14 | N + 14 | N + 2 | N + 20 |
| VVMD32 | | VRZAG 62 | | VRZAG 79 | |
| N + 5 | N + 37 | N + 20 | N + 20 | N + 8 | N + 22 |

Use international coding based on “N” (see European project GENRES 081—A basis for the preservation and utilization of Vitis genetic resources)
Phenological characteristics (in Rauscedo, Italy):
   Germination.—April 6.
   Flowering.—June 2.
   Veraison (change of color).—August 4.
   Maturation.—August 29.
Use: Wine grape.
Disease/pest resistance: Resistance to downy mildew and powdery mildew.

We claim:

1. A new and distinct variety of Vitis vinifera L. plant substantially as illustrated and described herein.
